(19) United States
(12) Patent Application Publication
(10) Pub. No.: US 2023/0074209 A1
(43) Pub. Date: Mar. 9, 2023

(54) METHOD AND DEVICE FOR PROVIDING DRUG RECOMMENDATION

(71) Applicant: Cancerfree biotech Ltd., Taipei City (TW)

(72) Inventors: Po-Han CHEN, Taipei City (TW); Shih-Pei Wu, Yilan County (TW); Yi-Hsuan Chen, Miaoli County (TW)

(21) Appl. No.: 17/502,309

(22) Filed: Oct. 15, 2021

(30) Foreign Application Priority Data
Aug. 30, 2021 (TW) 110131968

(51) Int. Cl. G16H 70/40; G16C 20/30; C12N 5/09; G16H 20/10

(52) U.S. Cl. CPC G16H 70/40; G16C 20/30; C12N 5/0693; G16H 20/10

(57) ABSTRACT

A method for providing drug recommendation includes: calculating a plurality of relative cell viabilities respectively corresponding to a plurality of candidate drugs based on viable cell counts of a patient's circulating tumor cell (CTC)-derived organoid cultures and viable cell counts of the CTC-derived organoid cultures respectively reacted with the candidate drugs; and providing drug recommendation of the candidate drugs based on the relative cell viabilities. A device for providing drug recommendation is also provided.

### CROSS-REFERENCE TO RELATED APPLICATION

This application claims priority to Taiwan Application Serial Number 110131968, filed Aug. 30, 2021, which is herein incorporated by reference.

### FIELD OF THE INVENTION

The present disclosure relates to a method and a device for providing drug recommendation, and in particular to a method and a device for providing drug recommendation based on individual differences of patients.

### BACKGROUND OF THE INVENTION

Pancreatic ductal adenocarcinoma (PDAC) accounts for more than 85% of all pancreatic cancers, and is one of the most aggressive forms of solid malignancy currently known, and has a very poor prognosis, with 5-year survival rates of around 10%. At initial diagnosis, approximately 80% to 90% of patients have unresectable disease, and more than 50% have metastatic disease. In such cases, systemic chemotherapy represents the standard of care, and progression-free survival (PFS) is usually limited to 3 months to 6 months. Therefore, there is an urgent need for a method that can accurately screen drugs used for systemic chemotherapy in order to improve the therapeutic effect of the systemic chemotherapy.

### SUMMARY OF THE INVENTION

The present disclosure provides a method for providing drug recommendation, which includes: calculating a plurality of relative cell viabilities respectively corresponding to a plurality of candidate drugs based on viable cell counts of a patient's circulating tumor cell (CTC)-derived organoid cultures and viable cell counts of the CTC-derived organoid cultures respectively reacted with the candidate drugs; and providing drug recommendation of the candidate drugs based on the relative cell viabilities.

In some embodiments of the present disclosure, providing the drug recommendation of the candidate drugs based on the relative cell viabilities includes: converting the relative cell viabilities into a plurality of scores respectively corresponding to the candidate drugs using at least one weighting process; and providing the drug recommendation of the candidate drugs based on the scores.

In some embodiments of the present disclosure, the method further includes: providing the weighting process, including: providing at least two relative cell viability intervals; and giving the at least two relative cell viability intervals with two different scores, respectively.

In some embodiments of the present disclosure, the method further includes: providing a first weighting process corresponding to at least one of the candidate drugs, including: providing at least two first relative cell viability intervals; and giving the at least two first relative cell viability intervals with two different first scores, respectively; and providing a second weighting process corresponding to at least another of the candidate drugs, including: providing at least two second relative cell viability intervals; and giving the at least two second relative cell viability intervals with two different second scores, respectively, in which a range of each of the first relative cell viability intervals is different from a range of each of the second relative cell viability intervals.

In some embodiments of the present disclosure, providing the drug recommendation of the candidate drugs based on the scores includes: recommending use of at least one or a combination of the candidate drugs based on the scores and a predicted effective total score range.

In some embodiments of the present disclosure, the score corresponding to the at least one of the candidate drugs or sum of the scores corresponding to the combination of the candidate drugs falls into the predicted effective total score range.

In some embodiments of the present disclosure, the method further includes: obtaining a liquid biopsy of the patient; isolating circulating tumor cells (CTCs) from the liquid biopsy; and expanding the CTCs to obtain the CTC-derived organoid cultures.

In some embodiments of the present disclosure, the patient is a patient with pancreatic cancer, lung cancer, breast cancer, sarcoma, melanoma, liver cancer, esophageal cancer, colorectal cancer, nasopharyngeal cancer, or brain cancer.

The present disclosure also provides device for providing drug recommendation, which includes a calculation module, a weighting processing module and a suggestion module. The calculation module is configured to calculate a plurality of relative cell viabilities respectively corresponding to a plurality of candidate drugs based on viable cell counts of a patient's CTC-derived organoid cultures and viable cell counts of the CTC-derived organoid cultures respectively reacted with the candidate drugs. The weighting processing module is connected to the calculation module and configured to convert the relative cell viabilities into a plurality of scores respectively corresponding to the candidate drugs using at least one weighting process. The suggestion module is connected to the weighting processing module and configured to provide drug recommendation of the candidate drugs based on the scores.

In some embodiments of the present disclosure, the suggestion module is configured to recommend use of at least one or a combination of the candidate drugs based on the scores and a predicted effective total score range.

### BRIEF DESCRIPTION OF THE DRAWINGS

FIG. 1 is a flowchart of a method for providing drug recommendation according to some embodiments of the present disclosure.

FIG. 2 is a functional module diagram of a device for providing drug recommendation according to some embodiments of the present disclosure.

### DETAILED DESCRIPTION

In order to make the description of the present disclosure more detailed and complete, the following provides an illustrative description for the implementation of the present disclosure and specific embodiments; but this is not the only way to implement or use the specific embodiments of the present disclosure. The embodiments disclosed below can be combined or substituted with each other under beneficial circumstances, and other embodiments can also be added to an embodiment without further description.

As described in the related art, pancreatic ductal adenocarcinoma is one of the most aggressive forms of solid malignancy currently known, and has a very poor prognosis, with very short progression-free survival of patients. Therefore, there is an urgent need for a method that can accurately screen drugs used for systemic chemotherapy in order to improve a therapeutic effect of the systemic chemotherapy.

However, there are very few biomarkers to inform treatment decisions for pancreatic ductal adenocarcinoma, and it is challenging to collect tumor samples for genomic or drug sensitivity testing. Therefore, the present disclosure uses a liquid biopsy containing circulating tumor cells (CTCs) served as a source of tumor tissue, which is relatively easy to obtain and is minimally invasive.

Although the CTCs can be isolated from the liquid biopsy, it is not easy to expand the CTCs in vitro. Therefore, after the CTCs are isolated from the liquid biopsy, a biomimetic cell culture system is used to efficiently expand the CTCs in vitro and thus to form CTC-derived organoid cultures. The biomimetic cell culture system does not use size elimination or epithelial marker capture methods, thereby preserving the heterogeneity of the CTCs to ensure that the CTC-derived organoid cultures are more representative of actual tumor conditions.

The present disclosure conducts drug sensitivity testing and calculations of relative cell viabilities on the CTC-derived organoid cultures, and even further performs a weighting process to provide patients with more accurate and personalized drug recommendation to improve a success rate of cancer treatment. Various embodiments of a method and a device for providing the drug recommendation of the present disclosure will be described in detail below.

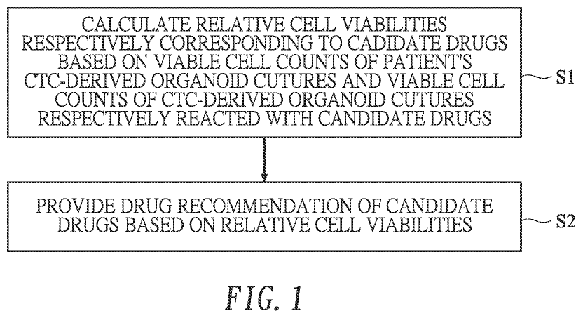

FIG. 1 is a flowchart of a method for providing drug recommendation according to some embodiments of the present disclosure. As shown in FIG. 1, the method includes following steps S1 and S2. It should be noted that the method for providing the drug recommendation of the present disclosure can be applied to patients with pancreatic cancer (e.g., advanced pancreatic ductal adenocarcinoma) as well as other cancer patients, such as lung cancer, breast cancer, sarcoma, melanoma, liver cancer, esophageal cancer, colorectal cancer, nasopharyngeal cancer, or brain cancer patients.

Step S1: a plurality of relative cell viabilities corresponding to a plurality of candidate drugs are calculated based on viable cell counts of a patient's CTC-derived organoid cultures and viable cell counts of the CTC-derived organoid cultures respectively reacted with the candidate drugs.

In some embodiments, first, a liquid biopsy of the patient is obtained, and CTCs are then isolated from the liquid biopsy, and the CTCs are then expanded to obtain the CTC-derived organoid cultures. The liquid biopsy is, for example, a blood sample, such as a peripheral venous blood sample. In one example, the CTCs were isolated from the liquid biopsy, and the biomimetic cell culture system was then used to expand the CTCs in vitro to form the CTC-derived organoid cultures. In one example, Ficoll-Paque centrifugation was used to isolate peripheral blood mononuclear cells (PBMCs) containing the CTCs from the blood sample. The cells were then seeded onto a substrate of binary colloidal crystal (BCC) containing silica and polymethyl methacrylate (PMMA), and cultured in the biomimetic cell culture system for three weeks, with replacement of culture medium every four days. The expansion of the organoid cultures could be monitored by optical microscopy, and the presence of the CTCs could be confirmed by EpCAM and CD45 immunofluorescence staining. The CTC-derived organoid cultures were positive for EpCAM and negative for CD45 in the immunofluorescence staining

In one example, the CTC-derived organoid cultures were resuspended in a culture medium, and then divided into aliquots and loaded to 96-well plates for drug sensitivity assays. The aliquots were divided into a control group (not reacted with a drug) and drug test groups (respectively reacted with different drugs). For advanced pancreatic ductal adenocarcinoma, gemcitabine, 5-fluorouracil (5-FU), erlotinib, irinotecan, olaparib, oxaliplatin, paclitaxel, palbociclib and trametinib were selected as candidate drugs and tested against the CTC-derived organoid cultures at concentrations according to the clinical pharmacokinetics of each of the drugs, in order to mimic the effect of a clinical dose in patients. Different drug treatments were respectively added to each of the wells in the 96-well plates, after which cultures were continued to co-culture for one week. At the end of the assay period, viable cell counts were measured using cytosolic adenosine triphosphate (ATP) abundance (CellTiter Glo, Promega, Madison Wis., USA) via a luminometer (Promega). The relative cell viabilities of the CTC-derived organoid cultures respectively corresponding to the 9 candidate drugs were calculated by the formula: relative cell viability=(viable cell counts of the drug test group/viable cell counts of the control group)×100%. Lower cell viability following application of test treatments was indicative of greater drug sensitivity and better anti-tumor effect. However, the present disclosure is not limited to the above example, and other test methods or calculation methods can be used to obtain the relative cell viabilities.

Step S2: drug recommendation of the candidate drugs is provided based on the relative cell viabilities.

In some embodiments, if the relative cell viabilities of one or more of the candidate drugs is less than 30%, or less than or equal to 29.9%, it is recommended to use the one or more drugs. In one example, a 61-year-old female patient presenting with locally advanced pancreatic cancer experienced intraperitoneal disease progression after concurrent chemoradiotherapy and maintenance weekly gemcitabine. Samples of CTCs from the patient were obtained in October 2018. During this period, her clinical performance continued to deteriorate, and clinical images indicated progression of intraperitoneal diseases despite salvage treatment with three courses of FOLFIRI. CTC sensitivity profiling showed resistance to 5-FU, oxaliplatin, paclitaxel, and irinotecan, and the findings were consistent with the clinical disease course. However, erlotinib was found to suppress CTC proliferation with viability value at 27.8%, which means it could inhibit the proliferation of CTCs. Therefore, She received erlotinib combined with gemcitabine at the discretion of the treating physician from December 2018, and enjoyed a clinical response for 4 months with improved performance status and decreased serum level of CA199. Her primary tumor biopsy was retrospectively analyzed, and Sanger sequencing revealed an epidermal growth factor receptor (EGFR) exon 20 mutation. It has been reported that erlotinib in combination with gemcitabine is active in patients with EGFR-mutated pancreatic cancer, with disease control rates of 64%. A combination of cell sensitivity profiling of the CTC-derived organoid cultures and tumor sequencing can provide response biomarkers, as has been observed in this case.

In some embodiments, if the relative cell viabilities of one or more drugs of the candidate drugs is less than 20%, or less than or equal to 19.9%, it is recommended to use the one or more drugs. In one example, a 57-year-old male patient was admitted for occult gastrointestinal bleeding on May 9, 2019, and was subsequently diagnosed with metastatic pancreatic cancer. Computer tomography (CT) scanning identified a 4.7-cm pancreatic tail tumor and 15 liver metastases. Radiotherapy (44 Gy/20 Fx) was initiated for the patient on May 16, 2019, and the patient also received 12 cycles of chemotherapy with FOLFIRINOX beginning on May 28, 2019. In August 2019, an abdominal CT scan revealed stable disease (SD) for the pancreatic mass and partial response (PR) for the liver metastases. However, a follow-up CT scan in November 2019 indicated progressive disease (PD) in the pancreatic tumor, together with liver, bone, and peritoneal seeding. A 20-mL liquid biopsy was collected from the patient in October 2019 prior to the follow-up scan, and CTC-derived organoid cultures expansion were successfully achieved in the biomimetic cell culture system. Drug sensitivity testing indicated that relative cell viability following olaparib treatment was 16.7%. Genetic testing was subsequently conducted on a biopsy taken from this patient in January 2020, and the results showed that the patient carried mutations in both the ATM (ataxia telangiectasia mutated) and BRIP1 (BRCA1-interacting protein 1) genes. ATM and BRIP1 work with BRCA1 (breast cancer 1) to repair DNA damage through the homologous recombination pathway, and tumor cells with defects in this pathway are dependent on the polyadenosine 5′-diphosphoribose [poly-(ADP-ribose)] polymerization (PARP) pathway for DNA repair. Olaparib is a PARP inhibitor that can block this pathway and disrupt DNA repair in tumor cells. However, considering that mutations in BRCA1 and the homologous recombination pathway are rarely seen in pancreatic ductal adenocarcinoma, the potential effectiveness of olaparib as predicted by the drug sensitivity test and subsequently corroborated in the genetic analysis report suggest that the drug sensitivity test of the CTC-derived organoid cultures of the present disclosure has the potential to reveal the presence of key mutations or other genetic features that would increase the efficacy of targeted treatments.

In some embodiments, the step S2 includes: converting the relative cell viabilities into a plurality of scores respectively corresponding to the candidate drugs using at least one weighting process (step S2-1); and providing the drug recommendation of the candidate drugs based on the scores (step S2-2).

In some embodiments, the method further includes: before the step 2-1, providing the weighting process, which includes: providing at least two relative cell viability intervals; and giving the at least two relative cell viability intervals with two different scores, respectively.

In one example, a receiver operating characteristic (ROC) curve using Youden's index was used to provide three relative cell viability intervals, and different three scores were given to the three relative cell viability intervals, respectively, as listed in Table 1. In one example, the relative cell viabilities corresponding to the nine candidate drugs in the above-mentioned drug sensitivity test were converted into nine scores using Table 1.

TABLE 1

| Relative Cell Viability Interval | Score |
|---|---|
| 0.0-29.9% | 4 |
| 30.0-69.9% | 1 |
| 70.0-100.0% | 0 |

In some embodiments, the method further includes: before the step 2-1, providing a first weighting process corresponding to at least one of the candidate drugs, which includes: providing at least two first relative cell viability intervals; and giving the at least two first relative cell viability intervals with two different first scores, respectively; and providing a second weighting process corresponding to at least another of the candidate drugs, which includes: providing at least two second relative cell viability intervals; and giving the at least two second relative cell viability intervals with two different second scores, respectively, in which a range of each of the first relative cell viability intervals is different from a range of each of the second relative cell viability intervals.

In an example, in addition to the first weighting process as listed in Table 1, a second weighting process is also provided, as listed in Table 2. At least one of the candidate drugs corresponds to the first weighting process, and its relative cell viability is converted into a score using Table 1. At least another of the candidate drugs corresponds to the second weighting process, and its relative cell viability is converted into a score using Table 2. The ranges of the three intervals (0.0 to 29.9%, 30.0% to 69.9%, and 70.0% to 100%) of the first weighting process and the ranges of the two intervals (0.0 to 14.9% and 15.0% to 100%) of the second weighting process were different.

TABLE 2

| Relative Cell Viability Interval | Score |
|---|---|
| 0.0-14.9% | 4 |
| 15.0-100.0% | 0 |

In practical applications, amounts, ranges, and/or corresponding scores of the relative cell viability intervals can be adjusted according to the feedback of the clinical response of multiple patients, so that subsequent personalized drug recommendation can be more accurate. The amounts, ranges, and/or corresponding scores of the relative cell viability intervals can also be adjusted according to characteristics of the drugs or other factors.

In some embodiments, the step S2-2 includes: recommending use of at least one or a combination (e.g., a combination of two or more drugs) of the candidate drugs based on the scores and a predicted effective total score range.

The predicted effective total score range can be obtained in the following method. First, the drug sensitivity test results of multiple patients were obtained, and the relative cell viabilities were calculated, and scores respectively corresponding to the candidate drugs were obtained by the one or more weighting processes. Afterwards, a prospective utility analysis was carried out to compare sum of all scores for drugs received by each of the patients during a treatment period (also can be called as cytotoxicity score of received treatments, CTS) and clinical response of each of the patients after receiving the treatment to confirm whether the prospective utility analysis is statistically significant.

In one example, clinical response to treatment in patients was evaluated using Response Evaluation Criteria in Solid Tumors (RECIST) version 1.1 criteria. The clinical response was divided into complete response (CR), partial response (PR), stable disease (SD) and progressive disease (PD). However, the present disclosure is not limited to the above example, and the clinical response can also be classified according to the latest version of Response Evaluation Criteria in Solid Tumors or other evaluation criteria.

In one example, a total of 41 liquid biopsies were collected from 31 patients with pancreatic cancer, and 87.8% of liquid biopsies were from patients with Stage 4 disease. PBMCs with enriched CTCs isolated from the liquid biopsy were then seeded onto a substrate of binary colloidal crystal and cultured for 3 weeks. It was ascertained that CTC-derived organoid cultures were successfully cultured from 87.8% (36/41) of biopsies. Next, drug sensitivity tests were performed, and relative cell viabilities were calculated according to the above-mentioned example, and scores respectively corresponding to the candidate drugs were obtained using the weighting process shown in Table 1. Afterwards, a prospective utility analysis was carried out, and sum of all scores for drugs received by the patient during the treatment period (CTS) (for at least 6 weeks in duration during the 3-month period after liquid biopsy) and the clinical response after receiving the treatment are listed in Table 3, and the results are summarized in Table 4.

TABLE 3

| Patient | | | AJCC | CTC | | |
|---|---|---|---|---|---|---|
| No. | Sex | Age | Stage | Expansion | CTS | Clinical Response |
| 01 | F | 61 | 4 | N | N/A | Partial Response |
| | | 61 | 4 | Y | 1 | Progressive Disease |
| | | 61 | 4 | Y | N/A | Progressive Disease |
| 02 | F | 64 | 4 | Y | 4 | Stable Disease |
| | | 64 | 4 | Y | 1 | Stable Disease |
| | | 64 | 4 | Y | 5 | Progressive Disease |
| 03 | F | 50 | 4 | Y | 0 | Progressive Disease |
| 04 | M | 77 | 4 | Y | 0 | Stable Disease |
| | | 79 | 4 | Y | N/A | Progressive Disease |
| 05 | M | 51 | 4 | N | N/A | Progressive Disease |
| 06 | M | 64 | 4 | Y | 0 | Stable Disease |
| 07 | F | 57 | 4 | Y | 2 | Stable Disease |
| 08 | F | 55 | 4 | Y | N/A | Progressive Disease |
| 09 | M | 67 | 4 | N | N/A | N/A |
| 10 | M | 56 | 4 | Y | 0 | Stable Disease |
| 11 | M | 60 | 4 | Y | 1 | Stable Disease |
| 12 | M | 57 | 4 | Y | 3 | Progressive Disease |
| | | 58 | 4 | Y | 0 | Progressive Disease |
| 13 | F | 67 | 4 | Y | 1 | Progressive Disease |
| 14 | F | 60 | 4 | Y | 1 | Stable Disease |
| 15 | F | 77 | 2 | Y | 1 | Stable Disease |
| 16 | M | 48 | 4 | Y | 4 | Stable Disease |
| 17 | M | 70 | 4 | N | N/A | Progressive Disease |
| 18 | M | 68 | 4 | Y | 1 | Progressive Disease |
| 19 | F | 49 | 4 | Y | 1 | Progressive Disease |
| | | 49 | 4 | Y | N/A | Progressive Disease |
| 20 | F | 83 | 3 | Y | 1 | Progressive Disease |
| 21 | F | 63 | 4 | Y | 0 | Stable Disease |
| 22 | M | 84 | 3 | Y | N/A | Progressive Disease |
| 23 | F | 61 | 4 | N | N/A | Stable Disease |
| 24 | M | 70 | 3 | Y | 8 | Progressive Disease |
| | | 70 | 4 | Y | 8 | Stable Disease |
| 25 | M | 79 | 4 | Y | 5 | Progressive Disease |
| 26 | M | 71 | 4 | Y | 1 | Progressive Disease |
| | | 71 | 4 | Y | 0 | Progressive Disease |
| 27 | F | 49 | 3 | Y | 4 | Partial Response |
| | | 49 | 4 | Y | N/A | Progressive Disease |
| 28 | F | 26 | 4 | Y | 5 | Stable Disease |
| 29 | M | 80 | 4 | Y | 4 | Stable Disease |
| 30 | F | 70 | 4 | Y | 5 | Partial Response |
| 31 | F | 63 | 4 | Y | 5 | Partial Response |

AJCC Stage: American Joint Committee on Cancer Staging Manual
N/A: not available (CTC expansion was unsuccessful (fail expansion), or the patient received the drug but there was no corresponding drug sensitivity result available (unmatched), or the patient received radiotherapy)

TABLE 4

| | Progressive | Stable | Partial | Complete |
|---|---|---|---|---|
| CTS | Disease | Disease | Response | Response |
| N/A | 8 | 1 | 1 | 0 |
| 0 | 3 | 4 | 0 | 0 |
| 1 | 6 | 4 | 0 | 0 |
| 2 | 0 | 1 | 0 | 0 |
| 3 | 1 | 0 | 0 | 0 |
| 4 | 0 | 3 | 1 | 0 |
| 5 | 2 | 1 | 2 | 0 |
| 6 | 0 | 0 | 0 | 0 |
| 7 | 0 | 0 | 0 | 0 |
| 8 | 1 | 1 | 0 | 0 |
| 9 | 0 | 0 | 0 | 0 |

N/A: not available

In one example, sums of all scores for drugs respectively received by the patients during the treatment period (CTS) were classified into two groups, i.e., less than 4 and greater than or equal to 4, and the clinical responses after the treatment were classified into a treatment ineffective group (e.g., “progressive disease” and “stable disease”) and a treatment effective group (e.g., “partial response” and “complete response”), as listed in Table 5. However, the present disclosure is not limited to the above-mentioned example, and it is also considered to classify “stable disease”, “partial response” and “complete response” as the treatment effective group and to classify “progressive disease” as the treatment ineffective group.

TABLE 5

| | Stable or | Partial or |
|---|---|---|
| Utility | Progressive Disease | Complete Response |
| CTS <4 | 18 | 0 |
| CTS ≥4 | 7 | 3 |

The data in Table 5 was calculated by the two-tailed Fisher's exact test with a p value of 0.0406, which was less than 0.05, which meant that the 2×2 contingency table in Table 5 was statistically significant, so it could be determined that a predicted effective total score range was greater than or equal to 4. In another aspect, an odds ratio of the data in Table 5 was 16.0588. However, the present disclosure is not limited to the above example. The classification of the sums of all scores for drugs respectively received by the patients during the treatment period and the classification of the clinical responses can be adjusted appropriately, and those can be adjusted continuously after collecting data of more patients, so that 2×2 contingency table will be more statistically significant.

According to the scores respectively corresponding to the candidate drugs obtained from the results of the patient's drug sensitivity test and the predicted effective total score range (i.e., the total score range is greater than or equal to 4), the physician can advise the patient to use at least one or a combination of the candidate drugs. In some embodiments, the score corresponding to the at least one of the candidate drugs or sum of the scores corresponding to the combination of the candidate drugs falls into the predicted effective total score range (i.e., the total score range is greater than or equal to 4). In one example, the relative cell viability of erlotinib for the 61-year-old female patient mentioned above was 27.8%. According to Table 1, erlotinib corresponds to the score of 4, which falls within the predicted effective total score range, so it is recommended that the physician consider using erlotinib to treat the patient. In one example, the relative cell viability of olaparib for the 57-year-old male patient mentioned above was 16.7%. According to Table 1, olaparib corresponds to the score of 4, which falls within the predicted effective total score range, so it is recommended that the physician consider using olaparib to treat the patient.

It can be seen from the above that the CTC-derived organoid cultures cultivated using the biomimetic cell culture system has high culture efficiency and very short expansion time. In addition, extraction of CTCs directly from the blood sample prevents both the loss of non-epithelial CTCs and the elimination of smaller and more deformable CTCs when physical techniques are used. Importantly, the biomimetic cell culture system can also conduct high-throughput drug sensitivity screening for several different anticancer treatments at the same time, and this has the potential to be used for early-stage cancer detection, optimal treatment selection, treatment efficacy monitoring, and effective treatment screening in case of relapse or treatment failure.

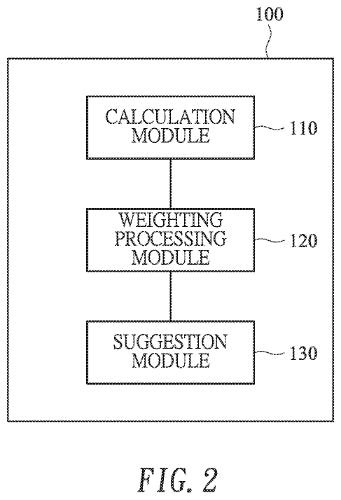

The present disclosure also provides a device for providing drug recommendation. FIG. 2 is a functional module diagram of a device for providing drug recommendation according to some embodiments of the present disclosure. As shown in FIG. 2, the device 100 includes a calculation module 110, a weighting processing module 120 and a suggestion module 130.

The calculation module 110 is configured to calculate a plurality of relative cell viabilities corresponding to a plurality of candidate drugs based on viable cell counts of a patient's CTC-derived organoid cultures and viable cell counts of the CTC-derived organoid cultures respectively reacted with the candidate drugs. That is, the calculation module 110 can receive the viable cell counts of a control group and those of drug test groups to calculate the relative cell viabilities of the CTC-derived organoid cultures respectively corresponding to the candidate drugs.

The weighting processing module 120 is connected to the calculation module 110. The weighting processing module 120 is configured to convert the relative cell viabilities into a plurality of scores respectively corresponding to the candidate drugs using at least one weighting process. The weighting process can refer to Table 1 or Table 2, so it is not repeated herein.

The suggestion module 130 is connected to the weighting processing module 120. The suggestion module 130 is configured to provide drug recommendation of the candidate drugs based on the scores. For example, recommendation of use of at least one or a combination of the candidate drugs is provided based on the scores.

In some embodiments, the suggestion module 130 is configured to provide the drug recommendation of the candidate drugs based on the scores and a predicted effective total score range. The predicted effective total score range can be obtained through the above-mentioned example, so it is not repeated herein.

In some embodiments, the suggestion module 130 is further connected to the calculation module 110 to directly obtain data such as relative cell viabilities, and thus to directly provide the drug recommendation of the candidate drugs through the data.

The above-mentioned embodiments are only illustrative of the principles and effects of the present disclosure, as well as explaining the technical features of the present disclosure, rather than limiting the scope of protection of the present disclosure. Anyone who is familiar with the technology can easily complete changes or equal arrangements without violating the technical principles and spirit of the present disclosure. All of them belong to the scope of the present disclosure.

What is claimed is:

1. A method for providing drug recommendation, comprising:
calculating a plurality of relative cell viabilities respectively corresponding to a plurality of candidate drugs based on viable cell counts of a patient's circulating tumor cell (CTC)-derived organoid cultures and viable cell counts of the CTC-derived organoid cultures respectively reacted with the candidate drugs; and
providing drug recommendation of the candidate drugs based on the relative cell viabilities.

2. The method of claim 1, wherein providing the drug recommendation of the candidate drugs based on the relative cell viabilities comprises:
converting the relative cell viabilities into a plurality of scores respectively corresponding to the candidate drugs using at least one weighting process; and
providing the drug recommendation of the candidate drugs based on the scores.

3. The method of claim 2, further comprising:
providing the weighting process, comprising:
providing at least two relative cell viability intervals; and
giving the at least two relative cell viability intervals with two different scores, respectively.

4. The method of claim 2, further comprises:
providing a first weighting process corresponding to at least one of the candidate drugs, comprising:
providing at least two first relative cell viability intervals; and
giving the at least two first relative cell viability intervals with two different first scores, respectively; and
providing a second weighting process corresponding to at least another of the candidate drugs, comprising:
providing at least two second relative cell viability intervals; and
giving the at least two second relative cell viability intervals with two different second scores, respectively, wherein a range of each of the first relative cell viability intervals is different from a range of each of the second relative cell viability intervals.

5. The method of claim 2, wherein providing the drug recommendation of the candidate drugs based on the scores comprises: recommending use of at least one or a combination of the candidate drugs based on the scores and a predicted effective total score range.

6. The method of claim 5, wherein the score corresponding to the at least one of the candidate drugs or sum of the scores corresponding to the combination of the candidate drugs falls into the predicted effective total score range.

7. The method of claim 1, further comprises:
obtaining a liquid biopsy of the patient;
isolating circulating tumor cells (CTCs) from the liquid biopsy; and
expanding the CTCs to obtain the CTC-derived organoid cultures.

8. The method of claim 1, wherein the patient is a patient with pancreatic cancer, lung cancer, breast cancer, sarcoma, melanoma, liver cancer, esophageal cancer, colorectal cancer, nasopharyngeal cancer, or brain cancer.

9. A device for providing drug recommendation, comprising:
a calculation module, configured to calculate a plurality of relative cell viabilities respectively corresponding to a plurality of candidate drugs based on viable cell counts of a patient's CTC-derived organoid cultures and viable cell counts of the CTC-derived organoid cultures respectively reacted with the candidate drugs;
a weighting processing module, connected to the calculation module and configured to convert the relative cell viabilities into a plurality of scores respectively corresponding to the candidate drugs using at least one weighting process; and
a suggestion module, connected to the weighting processing module and configured to provide drug recommendation of the candidate drugs based on the scores.

10. The device of claim 9, wherein the suggestion module is configured to recommend use of at least one or a combination of the candidate drugs based on the scores and a predicted effective total score range.
